(19) United States
(12) Patent Application Publication
(10) Pub. No.: US 2019/0200867 A1
(43) Pub. Date: Jul. 4, 2019

(54) OPTICAL FIBER SCANNER, ILLUMINATING DEVICE, AND OBSERVATION DEVICE

(71) Applicant: Olympus Corporation, Tokyo (JP)

(72) Inventors: Hirokazu Yokota, Tokyo (JP); Yasuaki Kasai, Saitama-shi (JP); Hiroshi Tsuruta, Sagamihara-shi (JP); Takashi Yasumi, Tokyo (JP)

(73) Assignee: Olympus Corporation, Tokyo (JP)

(21) Appl. No.: 16/295,618

(22) Filed: Mar. 7, 2019

Related U.S. Application Data
(63) Continuation of application No. PCT/JP2016/077167, filed on Sep. 14, 2016.

(51) Int. Cl. A61B 5/00; G02B 26/10

(52) U.S. Cl. CPC A61B 5/0062; A61B 2562/0242; G02B 26/103

(57) ABSTRACT

The technology is directed to an optical fiber scanner configured to be used in an observation device. The optical fiber scanner comprises an optical fiber guiding light therethrough. A vibration transmitting member is configured to transmit vibrations to the optical fiber. The vibration transmitting member includes a through hole defined therein through which the optical fiber extends. At least one piezoelectric element is disposed on an outer circumferential surface of the vibration transmitting member and vibrating a distal end portion of the optical fiber via the vibration transmitting member. A fixing member is disposed on a proximal end side of the vibration transmitting member. A tubular wire holding member includes a plurality of wires integrally attached for supplying a voltage to the piezoelectric element. The tubular wire holding member has a contact portion securing the plurality of wires to an outer peripheral surface of the piezoelectric element in contact therewith.

### CROSS-REFERENCE TO RELATED APPLICATION

This application is a continuation application of PCT Application No. PCT/JP2016/077167 filed on Sep. 14, 2016, which is hereby incorporated by reference in its entirety.

### TECHNICAL FIELD

The disclosed technology relates to an optical fiber scanner, an illuminating device, and an observation device.

### DESCRIPTION OF THE RELATED ART

There is known an optical fiber scanner in which the distal end of an optical fiber is vibrated by the vibrations of piezoelectric elements to scan a light beam emitted from the distal end of the optical fiber over a subject (see, for example, Japanese Patent Laid-Open No. 2010-97083 a.k.a, PTL 1).

The optical fiber scanner disclosed in PTL 1 includes an actuator in which the four piezoelectric elements are bonded to the optical fiber in a surrounding relation thereto and wires for supplying electric power are connected respectively to the piezoelectric elements.

As illustrated in FIG. 7, the optical fiber scanner disclosed in PTL 1 has the wires installed respectively on outer surfaces of the four piezoelectric elements. Since the intervals between the piezoelectric elements are small, it may be a hard task to attach the wires to the piezoelectric elements. If the conductive portions of two wires contact each other, causing a short circuit, then the actuator may not be able to operate normally.

### BRIEF SUMMARY OF EMBODIMENTS

The disclosed technology has been made in view of the above problems. It is an object of the disclosed technology to provide an optical fiber scanner, an illuminating device, and an observation device that allow wires to be positioned accurately with respect to piezoelectric elements for better assemblability of parts.

The disclosed technology is directed to an optical fiber scanner configured to be used in an observation device. The optical fiber scanner comprises an optical fiber guiding light therethrough. A vibration transmitting member is configured to transmit vibrations to the optical fiber. The vibration transmitting member includes a through hole defined therein through which the optical fiber extends. At least one piezoelectric element is disposed on an outer circumferential surface of the vibration transmitting member and vibrating a distal end portion of the optical fiber via the vibration transmitting member. A fixing member is disposed on a proximal end side of the vibration transmitting member and is holding the optical fiber in position. A tubular wire holding member includes a plurality of wires integrally attached thereof and extending longitudinally therealong for supplying a voltage to the piezoelectric element. The wire holding member is configured to cover at least a proximal end portion of the piezoelectric element. The tubular wire holding member has a contact portion securing the plurality of wires to an outer peripheral surface of the piezoelectric element in contact therewith.

The tubular wire holding member is made of a thermally shrinkable material or an optically shrinkable material. The optical fiber scanner further comprises an annular pressing member made of an elastic material that presses the contact portion against the outer peripheral surface of the piezoelectric element. The tubular wire holding member has a proximal end portion made of a material that is harder than other portion of the tubular wire holding member. The fixing member is integrally attached with the tubular wire holding member.

With this arrangement, the wires can be intimately held against and secured to the piezoelectric elements with ease simply when the wire holding member is thermally or optically shrunk after being positioned with respect to the piezoelectric elements.

With this arrangement, the contact portion of the wire holding member can be pressed against the piezoelectric element with the wires assembled therein under resilient forces of the pressing member, bringing the wires and the piezoelectric elements into more intimate contact with each other.

In the above aspect, at least a proximal end portion of the wire holding member may be made of a material that is harder than a distal end portion of the wire holding member.

With this arrangement, when the optical fiber scanner is assembled, the hard portion of the wire holding member may be gripped by a tool such as tweezers, so that the wire holding member can easily be attached to the outer peripheral surface of the piezoelectric element. Moreover, an elastic portion of the wire holding member is prevented from being deformed, and the wires incorporated in the wire holding member are prevented from being damaged.

In the above aspect, the fixing member may be integrally combined with the wire holding member.

With this arrangement, the fixing member that is integrally combined in advance with the wire holding member allows the optical fiber scanner to be assembled more easily than the optical fiber scanner where the fixing member is separately and independently included.

According to another aspect of the disclosed technology, there is provided an illuminating device including a light source, an optical fiber scanner referred to any described hereinbefore, the optical fiber scanner scanning light from the light source, and a condensing lens focusing light scanned by the optical fiber scanner.

According to still another aspect of the disclosed technology, there is provided an observation device including an illuminating device referred to hereinabove and a light detector detecting returning light from a subject when light is applied from the illuminating device to the subject.

The disclosed technology is advantageous in that wires can be positioned accurately with respect to piezoelectric elements for better assemblability of parts.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a longitudinal cross-sectional view illustrating an observation device and an illuminating device according to an embodiment of the disclosed technology.

FIG. 2A is a longitudinal cross-sectional view illustrating an optical fiber scanner according to the embodiment of the disclosed technology that is incorporated in the observation device illustrated in FIG. 1.

FIG. 2B is a perspective view of a wire holding member incorporated in the optical fiber scanner illustrated in FIG. 2A.

FIG. 2C is a transverse cross-sectional view taken along line A-A across the optical fiber scanner illustrated in FIG. 2A.

FIG. 3A is an enlarged partial view illustrating a modification of the optical fiber scanner illustrated in FIG. 2A.

FIG. 3B is a transverse cross-sectional view taken along line A-A across the optical fiber scanner illustrated in FIG. 3A.

FIG. 4 is an enlarged partial view illustrating a first modification of the wire holding member illustrated in FIG. 2B.

FIG. 5 is an enlarged partial view illustrating a second modification of the wire holding member illustrated in FIG. 2B.

FIG. 6 is an enlarged partial view illustrating a third modification of the wire holding member illustrated in FIG. 2B.

FIG. 7 is a longitudinal cross-sectional view illustrating an optical fiber scanner according to the related art.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

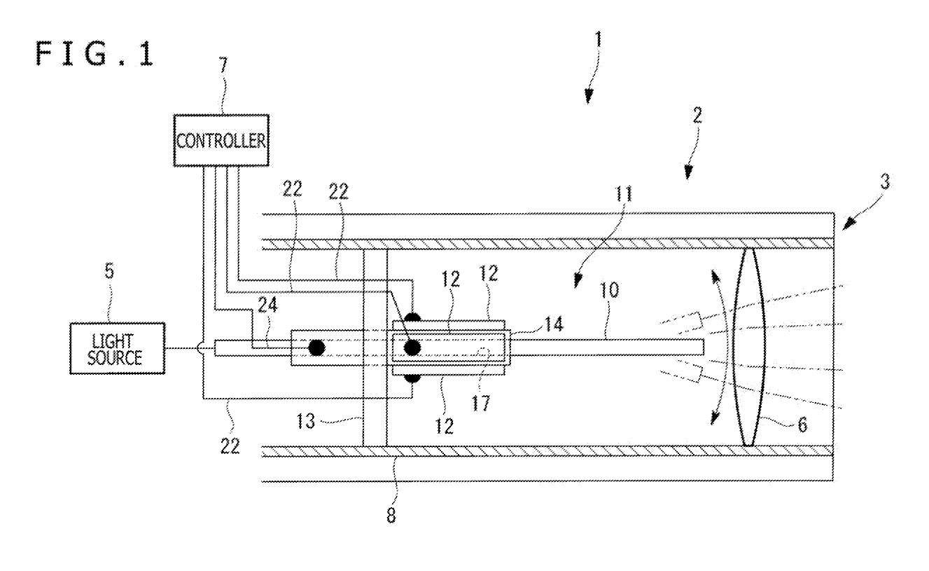

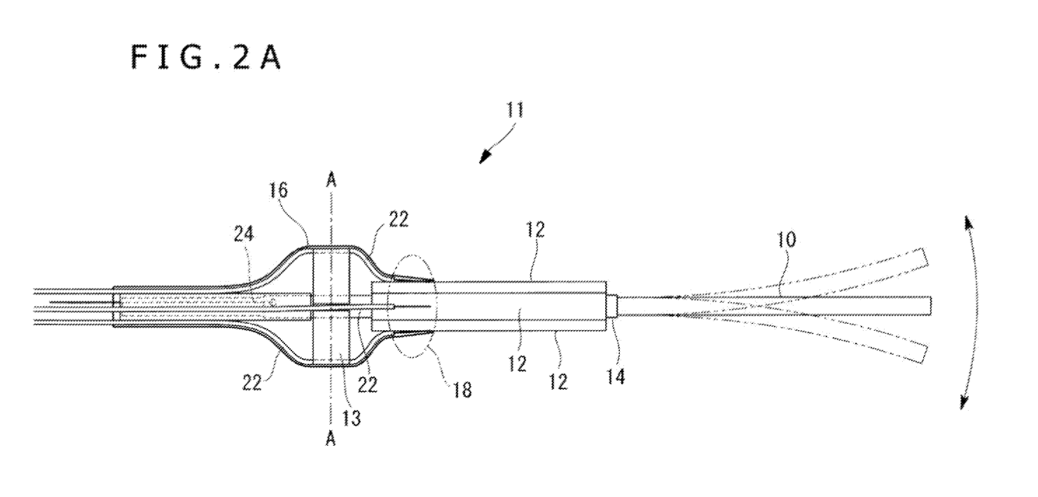

An optical fiber scanner 11, an illuminating device 2, and an observation device 1 according to an embodiment of the disclosed technology will be described hereinafter with reference to the drawings. FIG. 1 is a longitudinal cross-sectional view illustrating the observation device and the illuminating device according to the embodiment of the disclosed technology. FIG. 2A is a longitudinal cross-sectional view illustrating the optical fiber scanner according to the embodiment of the disclosed technology that is incorporated in the observation device illustrated in FIG. 1. FIG. 2B is a perspective view of a wire holding member incorporated in the optical fiber scanner illustrated in FIG. 2A. FIG. 2C is a transverse cross-sectional view taken along line A-A across the optical fiber scanner illustrated in FIG. 2A.

As illustrated in FIG. 1, the observation device 1 according to the present embodiment includes the illuminating device 2 applying illuminating light to a subject, a light detector 3 detecting returning light that returns from the subject, and a controller 7 controlling the illuminating device 2.

The illuminating device 2 according to the present embodiment includes a light source 5, the optical fiber scanner 11 scanning light from the light source 5, a condensing lens 6 disposed closer to the distal end of the illuminating device 2 than the optical fiber scanner 11, and focusing illuminating light emitted from the optical fiber scanner 11, and a frame 8 in the form of a slender tube that houses the optical fiber scanner 11 and the condensing lens 6 therein.

As illustrated in FIGS. 1 and 2A, the optical fiber scanner 11 includes an optical fiber 10 guiding the light from the light source 5 and emitting light from its distal end, an elastic member or vibration transmitting member 14 in the form of a quadrangular prism made of an electrically conductive, elastic material and having a through hole 17 defined therein with the optical fiber 10 extending therethrough, four piezoelectric elements 12 disposed on outer peripheral surfaces of the elastic member 14, and a fixing member 13 disposed on the proximal end side of the elastic member 14, and fixing the optical fiber 10 in position.

As illustrated in FIG. 2B, the optical fiber scanner 11 also includes a tubular wire-assembled tube or wire holding member 16 having wires 22 integrally combined therewith that extend longitudinally therealong, for supplying alternating voltages to the piezoelectric elements 12. Specifically, four wires 22, each corresponding to one of the four piezoelectric elements 12 disposed on the outer peripheral surfaces of the elastic member 14, are disposed on an inner circumferential surface of the wire-assembled tube 16 and extend longitudinally along the wire-assembled tube 16. The wires 22 are angularly spaced at 90° intervals in the circumferential directions of the wire-assembled tube 16. As illustrated in FIG. 2B, leads 25 are joined respectively to the distal ends of the wires 22, so that the wires 22 are electrically connected to respective outer peripheral surfaces of the corresponding piezoelectric elements 12 through the leads 25.

The voltages are applied to the respective piezoelectric elements 12 via the wires 22 disposed on the outer peripheral surfaces of the piezoelectric elements 12. Specifically, if it is assumed that the longitudinal axis of the optical fiber scanner 11 is referred to as a Z-axis and the two transverse axes of the optical fiber scanner 11 that are perpendicularly to each other and the Z-axis as an X-axis and a Y-axis, then an alternating voltage in an A phase is applied to the two piezoelectric elements 12 that are opposite each other along the X-axis and an alternating voltage in a B phase is applied to the two piezoelectric elements 12 that are opposite each other along the Y-axis. When the alternating voltages are applied to the piezoelectric elements 12, the elastic member 14 is flexurally vibrated. The vibrations of the elastic member 14 are transmitted to the optical fiber 10, displacing and vibrating the distal end of the optical fiber 10, from which the illuminating light is emitted, in directions that are transverse to the longitudinal axis of the optical fiber scanner 11.

Each of the piezoelectric elements 12 is made of a piezoelectric ceramics material such as lead zirconate titanate (PZT) or the like, for example. According to the present embodiment, as illustrated in FIG. 2C, the four piezoelectric elements 12, each in the form of a flat plate, are illustrated as being bonded to the four outer peripheral surfaces of the elastic member 14, which is of a square cross-sectional shape, by an adhesive 20. However, the piezoelectric elements 12 are not limited to four flat-plate piezoelectric elements. Rather, one or two piezoelectric elements formed to a U shape or an L shape that are capable of vibrating biaxially may be used, for example.

The elastic member 14 is in the form of a quadrangular prism having the through hole 17 defined therein with the optical fiber 10 extending therethrough along the longitudinal axis thereof. The elastic member 14 is made of an electrically conductive, elastic material. As illustrated in FIG. 2A, the elastic member 14 is disposed in an intermediate position along the longitudinal axis thereof from the distal end of the optical fiber 10 to the proximal end side thereof.

According to the present embodiment, the elastic member 14 is illustrated as being in the form of a quadrangular prism. However, the elastic member 14 is not limited to such a shape, but may be in the form of a polygonal prism or a hollow cylindrical shape as long as it allows the piezoelectric elements 12 capable of vibrating biaxially to be bonded thereto.

As illustrated in FIG. 2C, the fixing member 13 is a substantially annular, electrically conductive member having a central hole defined therein that is of a square cross-sectional shape. The fixing member 13 is secured by the adhesive 20 to the elastic member 14, more closely to the proximal end than the piezoelectric elements 12, fitted in the central hole. As illustrated in FIG. 2C, the fixing member 13 has four wire grooves 23 defined in an outer circumferential surface thereof and spaced at 90° intervals circumferentially, the wire grooves 23 extending along the longitudinal axis of the optical fiber scanner 11.

The outer circumferential surface of the fixing member 13 is fixed to an inner wall surface of the frame 8. The elastic member 14 is supported in a cantilevered fashion by the fixing member 13. The optical fiber 10 has a distal end portion supported in a cantilevered fashion with the distal end as a free end, by the elastic member 14.

A GND wire 24 is connected to a proximal end side of the elastic member 14.

As illustrated in FIGS. 2A and 2C, when the wire-assembled tube 16 is placed in covering relation to the fixing member 13 and extends from a proximal end side of the optical fiber 10 toward a distal end side thereof, the wires 22 disposed on the inner circumferential surface of the wire-assembled tube 16 are housed in the wire grooves 23 defined in the fixing member 13. The wires 22 are thus kept out of contact with each other.

The wire-assembled tube 16 has on a distal end thereof a contact region or contact portion 18 where the leads 25 joined to the distal ends of the wires 22 are held in contact with and fixed to outer peripheral surfaces of the corresponding piezoelectric elements 12.

The wire-assembled tube 16 is made of an elastic material. Specifically, the wire-assembled tube 16 may be a thermally shrinkable tube that shrinks when heated or optically shrinkable tube that shrinks when irradiated with a near-infrared ray or the like. The thermally shrinkable tube may be made of polyolefin rein, fluororesin, or silicone resin, for example. Also, the optically shrinkable tube may be made of isopropylacrylamide or the like, for example.

The wire-assembled tube 16 may be made of a transparent material such that the wires 22 and the piezoelectric elements 12 can be visually confirmed for their associated relationship when the wires 22 are to be positioned on the corresponding piezoelectric elements 12. Alternatively, markers for assisting in positioning the wires 22 and the piezoelectric elements 12 with respect to each other may be put on the outer circumferential surface of the wire-assembled tube 16.

The wires 22 and the GND wire 24 are made of an electrically conductive wire material such as copper, aluminum, or the like, for example.

Each of the wires 22 and the GND wire 24 has proximal ends connected to the controller 7, and the light source 5 is connected to the proximal end of the optical fiber 10.

The wires 22 are each covered with a thin insulating film that electrically insulates themselves from the surroundings, except for the leads 25 that are electrically connected to the piezoelectric elements 12. Since the wires 22, except for the leads 25, are covered with the insulating material, the wires 22 are less susceptible to external electric fields.

If the fixing member 13 is made of an insulating material, then the wires 22 may not be covered with a thin insulating film.

Operation of the optical fiber scanner 11, the illuminating device 2, and the observation device 1 according to the present embodiment, which are constructed as described hereinbefore, will be described hereinafter.

For observing a subject using the observation device 1 according to the present embodiment, the controller 7 is operated to supply illuminating light from the light source 5 to the optical fiber 10 and to apply alternating voltages having a predetermined drive frequency through the wires 22 to the piezoelectric elements 12.

When the A-phase alternating voltage is applied to the two piezoelectric elements 12 that are disposed opposite each other along the X-axis, one of the two piezoelectric elements 12 is expanded lengthwise, and the other piezoelectric element 12 is contracted lengthwise. The distal end of the optical fiber 10 is thus vibrated along the X-axis, linearly scanning the illuminating light emitted from the distal end of the optical fiber 10 along the X-axis on the subject.

Similarly, when the B-phase alternating voltage is applied to the two piezoelectric elements 12 that are disposed opposite each other along the Y-axis, one of the two piezoelectric elements 12 is expanded lengthwise, and the other piezoelectric element 12 is contracted lengthwise. The distal end of the optical fiber 10 is thus vibrated along the Y-axis, linearly scanning the illuminating light emitted from the distal end of the optical fiber 10 along the Y-axis on the subject.

Light returning from the subject is received by a receptive optical fiber, not shown, and its intensity is detected by the light detector 3. The controller 7 controls the light detector 3 to detect the returning light in synchronism with a scanning period of the illuminating light, and generates an image of the subject by associating a detected intensity of the returning light with a scanning position of the illuminating light. The generated image is output to a display, not shown, that displays the image thereon.

A process of manufacturing the optical fiber scanner 11 according to the present embodiment will be described hereinafter.

First, a predetermined range of the distal end of the optical fiber 10 is longitudinally inserted into the through hole 17 in the elastic member 14. Then, the four flat-plate piezoelectric elements 12 are bonded respectively to the four surfaces of the elastic member 14 in the form of a quadrangular prism by the adhesive 20. Thereafter, the elastic member 14 that is positioned more closely to the proximal end than the piezoelectric elements 12 is fitted in the central hole in the fixing member 13 and secured thereto by the adhesive 20. The elastic member 14 has its proximal end portion connected to the GND wire 24.

Next, the wire-assembled tube 16 made of a thermally shrinkable material and having elasticity is placed in covering relation to the fixing member 13 such that it extends from the proximal end side of the optical fiber toward the distal end side thereof. At this time, the wire-assembled tube 16 is expanded and placed over the outer circumferential surface of the fixing member 13, covering at least the outer peripheral surfaces of the proximal ends of the piezoelectric elements 12.

The wire-assembled tube 16 is circumferentially rotated and positioned to place the leads 25 of the four wires 22 that are disposed on the inner circumferential surface of the wire-assembled tube 16 and angularly spaced at 90° intervals in the circumferential directions, respectively on the outer peripheral surfaces of the four piezoelectric elements 12. At this time, as illustrated in FIG. 2C, since the four wire grooves 23 that extend along the longitudinal axis of the optical fiber scanner 11 are defined in the outer circumferential surface of the fixing member 13 and spaced at 90° intervals circumferentially, when the four wires 22 are each positioned so as to be housed in the wire grooves 23, each of the wires 22 are disposed above each of the outer circumferential surfaces of the piezoelectric elements 12. The wires 22 that are housed in the wire grooves 23 are prevented from contacting each other.

Thereafter, a front surface of the wire-assembled tube 16 is uniformly heated at a predetermined temperature to cause the wire-assembled tube 16 to thermally shrink radially. The wire-assembled tube 16 that is thus thermally shrunk joins the wires 22 to the piezoelectric elements 12. At this time, the inner circumferential surface of the wire-assembled tube 16 may be coated with a thin layer of the adhesive 20, and the adhesive 20 may be melted with heat to bond and secure the wires 22 to the piezoelectric elements 12 instantly. The elastic member 14 and the fixing member 13 may be made of a heat-resistant material to reduce thermal damage to the piezoelectric elements 12.

The wire-assembled tube 16 may be an optically shrinkable tube that shrinks when irradiated with light, instead of a thermally shrinkable tube.

As described hereinbefore, for securing the wires 22 in position to the respective piezoelectric elements 12, the tubular wire-assembled tube 16 with the wires 22 integrally pre-assembled therein is used to position each of the wires 22 accurately with respect to each of the piezoelectric elements 12. The wires 22 can thus be secured in position with ease. Therefore, the assemblability of the optical fiber scanner 11 is increased.

Since a thermally shrinkable tube or an optically shrinkable tube is used as the wire-assembled tube 16, the wires 22 can be intimately held against and secured to the piezoelectric elements 12 with ease when the wire-assembled tube 16 is thermally or optically shrunk.

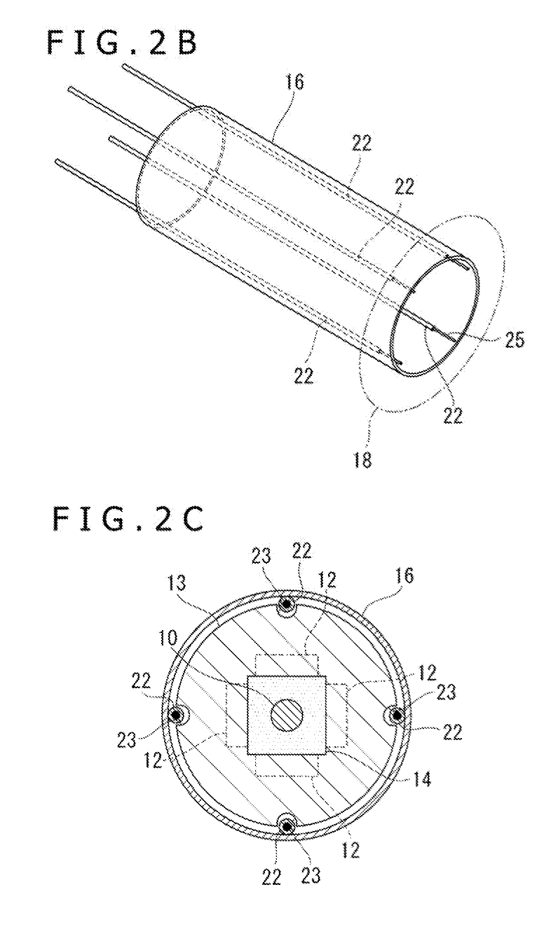

According to the present embodiment, the wire-assembled tube 16 is expanded and placed over the outer circumferential surface of the fixing member 13, thereby covering at least the outer peripheral surfaces of the proximal ends of the piezoelectric elements 12 to secure the wires 22 to the piezoelectric elements 12 in intimate contact therewith. However, as illustrated in FIGS. 3A and 3B, the wire-assembled tube 16 may be placed in advance over the outer peripheral surfaces of the piezoelectric elements 12 to position the wires 22 and the piezoelectric elements 12, after which the wire-assembled tube 16 may be shrunk to secure the wires 22 and the piezoelectric elements 12 to each other. Then, the wire-assembled tube 16 may be fitted into the central hole in the fixing member 13 and joined thereto by the adhesive 20. The modification may be employed appropriately by a design change depending on a purpose of the wire-assembled tube 16.

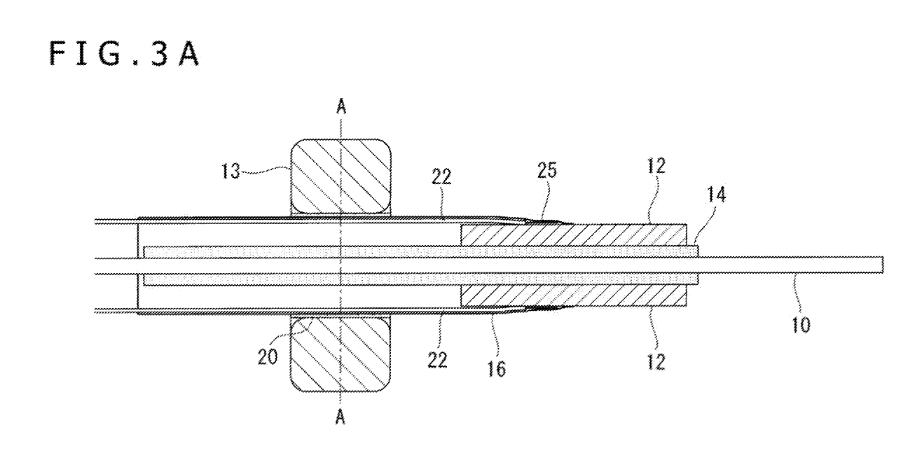

As illustrated in FIG. 4, the wire-assembled tube 16 may have a hard member 19 in a proximal end portion thereof, which is made of a hard metal or resin material that is harder than another portion of the wire-assembled tube 16.

With this arrangement, when the optical fiber scanner 11 is assembled, the hard member 19 of the wire-assembled tube 16 may be gripped by a tool such as tweezers, so that the other elastic portion of the wire-assembled tube 16 is prevented from being deformed or damaged to prevent the wires 22 on the wire-assembled tube 16 from being damaged or broken. Furthermore, the wire-assembled tube 16 may easily be attached to the outer peripheral surfaces of the piezoelectric elements 12.

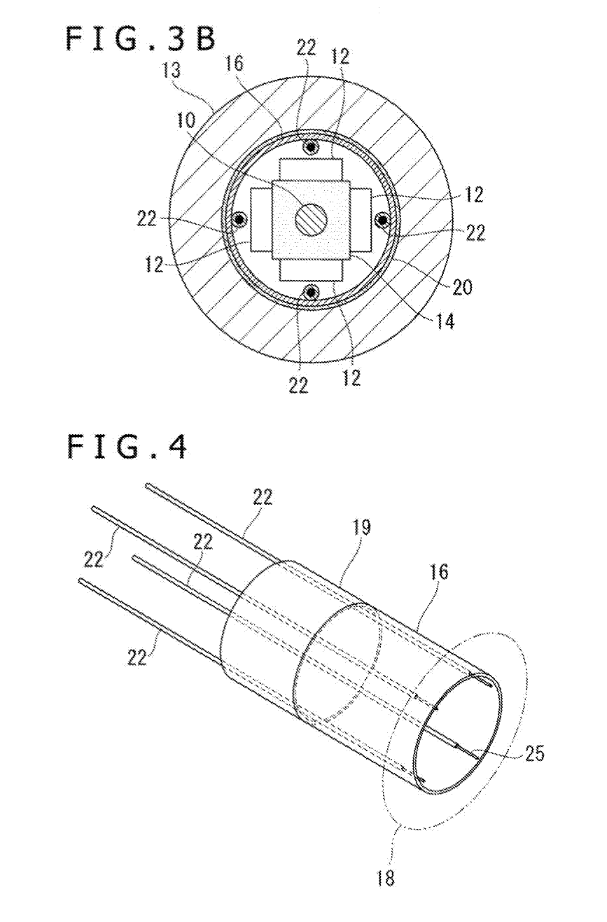

Moreover, as illustrated in FIG. 5, the wire-assembled tube 16 may further include an annular presser or pressing member 26 made of an elastic material such as rubber or the like for pressing the contact region 18 where the leads 25 are disposed against the outer peripheral surfaces of the piezoelectric elements 12.

With this arrangement, the contact region 18 of the wire-assembled tube 16 may be pressed against the piezoelectric elements 12 under resilient forces of the presser 26, bringing the wires 22 and the piezoelectric elements 12 into intimate contact with each other.

The piezoelectric elements 12 and the wires 22 may be fixed to each other more securely because the wires 22 are intimately held against and secured to the piezoelectric elements 12 by the shrinkage of the wire-assembled tube 16 and additionally by the elastic forces of the presser.

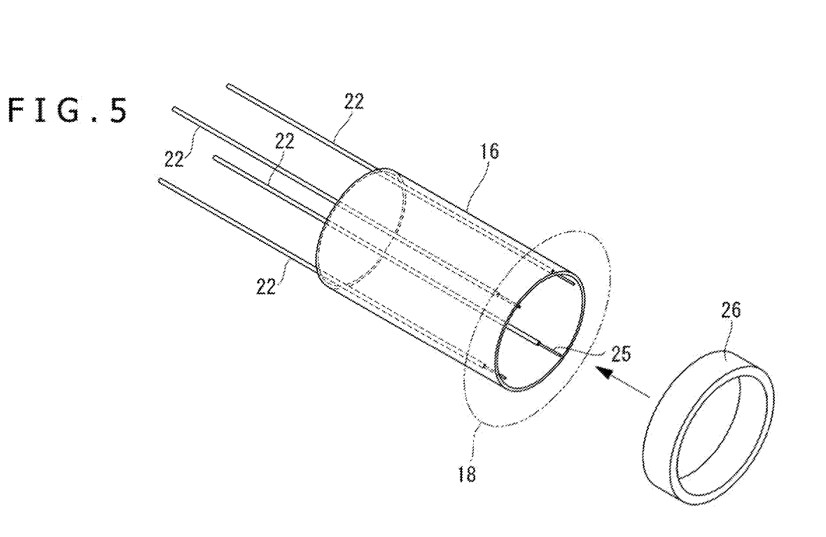

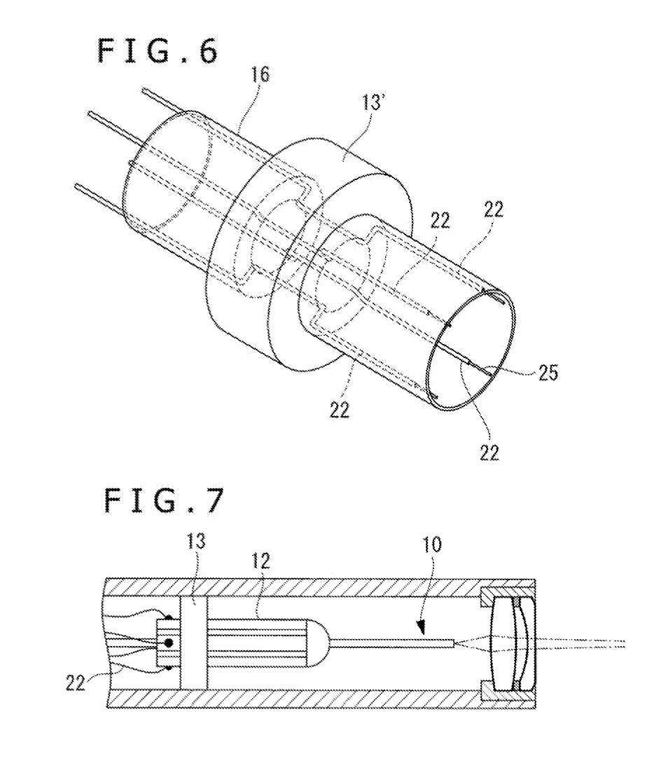

Furthermore, as illustrated in FIG. 6, a fixing member 13′ may be fitted in advance over the outer circumferential surface of the wire-assembled tube 16, so that they may be of an integrally combined structure.

With this arrangement, the optical fiber scanner 11 can be assembled more easily than the optical fiber scanner where the fixing member 13 is separately and independently included.

In sum, one aspect of the disclosed technology is directed to an optical fiber scanner configured to be used in an observation device. The optical fiber scanner comprises an optical fiber guiding light therethrough. A vibration transmitting member is configured to transmit vibrations to the optical fiber. The vibration transmitting member includes a through hole defined therein through which the optical fiber extends. At least one piezoelectric element is disposed on an outer circumferential surface of the vibration transmitting member and vibrating a distal end portion of the optical fiber via the vibration transmitting member. A fixing member is disposed on a proximal end side of the vibration transmitting member and is holding the optical fiber in position. A tubular wire holding member includes a plurality of wires integrally attached thereof and extending longitudinally therealong for supplying a voltage to the piezoelectric element. The wire holding member is configured to cover at least a proximal end portion of the piezoelectric element. The tubular wire holding member has a contact portion securing the plurality of wires to an outer peripheral surface of the piezoelectric element in contact therewith.

The tubular wire holding member is made of a thermally shrinkable material or an optically shrinkable material. The optical fiber scanner further comprises an annular pressing member made of an elastic material that presses the contact portion against the outer peripheral surface of the piezoelectric element. The tubular wire holding member has a proximal end portion made of a material that is harder than other portion of the tubular wire holding member. The fixing member is integrally attached with the tubular wire holding member.

Another aspect of the disclosed technology is directed to an illuminating device configured to be used in an observation device. The illuminating device comprises a light source. An optical fiber scanner comprises an optical fiber guiding light therethrough. A vibration transmitting member is configured to transmit vibrations to the optical fiber. The vibration transmitting member includes a through hole defined therein through which the optical fiber extends. At least one piezoelectric element is disposed on an outer circumferential surface of the vibration transmitting member and vibrating a distal end portion of the optical fiber via the vibration transmitting member. A fixing member is disposed on a proximal end side of the vibration transmitting member and holding the optical fiber in position. A tubular wire holding member includes a plurality of wires integrally attached thereof and extending longitudinally therealong for supplying a voltage to the piezoelectric element. The wire holding member is configured to cover at least a proximal end portion of the piezoelectric element. The tubular wire holding member has a contact portion securing the plurality of wires to an outer peripheral surface of the piezoelectric element in contact therewith. The optical fiber scanner scanning light from the light source and a lens focusing light scanned by the optical fiber scanner.

A further aspect of the disclosed technology is directed to an observation device comprises an illuminating device configured to apply illuminating light to a subject. A light detector is configured to detect returning light from the subject and a controller is used to electrically control the illuminating device. The illuminating device includes a light source. An optical fiber scanner is configured for scanning light from the light source. A lens focusing the light scanned by the optical fiber scanner. The optical fiber scanner includes an optical fiber guiding light therethrough. A vibration transmitting member is configured to transmit vibrations to the optical fiber. At least one piezoelectric element is disposed on an outer circumferential surface of the vibration transmitting member and vibrating a distal end portion of the optical fiber via the vibration transmitting member. A fixing member is disposed on a proximal end side of the vibration transmitting member and is holding the optical fiber in position. A tubular wire holding member includes a plurality of wires integrally attached thereof and extending longitudinally therealong for supplying a voltage to the least one piezoelectric element. The tubular wire holding member has a contact portion securing the plurality of wires to an outer peripheral surface of the piezoelectric element in contact therewith.

The vibration transmitting member includes a through hole defined therein through which the optical fiber extends. The wire holding member is configured to cover at least a proximal end portion of the piezoelectric element. The controller controls the light detector to detect the returning light in synchronism with a scanning period of the illuminating light and to generate an image of the subject by associating a detected intensity of the returning light with a scanning position of the illuminating light.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

### NUMERAL REFERENCE LIST

1 Observation device
2 Illuminating device
5 Light source
10 Optical fiber
11 Optical fiber scanner
12 Piezoelectric element
13, 13′ Fixing member
14 Elastic member or vibration transmitting member
16 Wire-assembled tube or wire holding member
22 Wire
26 Presser or pressing member

What is claimed is:

1. An optical fiber scanner configured to be used in an observation device, the optical fiber scanner comprising:
an optical fiber guiding light therethrough;
a vibration transmitting member configured to transmit vibrations to the optical fiber, the vibration transmitting member having a through hole defined therein through which the optical fiber extends;
at least one piezoelectric element disposed on an outer circumferential surface of the vibration transmitting member and vibrating a distal end portion of the optical fiber via the vibration transmitting member;
a fixing member disposed on a proximal end side of the vibration transmitting member and holding the optical fiber in position; and
a tubular wire holding member having a plurality of wires integrally attached thereof and extending longitudinally therealong for supplying a voltage to the piezoelectric element, the wire holding member configured to cover at least a proximal end portion of the piezoelectric element,
wherein the tubular wire holding member has a contact portion securing the plurality of wires to an outer peripheral surface of the piezoelectric element in contact therewith.

2. The optical fiber scanner of claim 1, wherein the tubular wire holding member is made of a thermally shrinkable material or an optically shrinkable material.

3. The optical fiber scanner of claim 1, further comprising:
an annular pressing member made of an elastic material that presses the contact portion against the outer peripheral surface of the piezoelectric element.

4. The optical fiber scanner of any of claim 1, wherein the tubular wire holding member has a proximal end portion made of a material that is harder than other portion of the tubular wire holding member.

5. The optical fiber scanner of claim 1, wherein the fixing member is integrally attached with the tubular wire holding member.

6. An illuminating device configured to be used in an observation device, the illuminating device comprising:
a light source;
an optical fiber scanner comprises an optical fiber guiding light therethrough;
a vibration transmitting member configured to transmit vibrations to the optical fiber, the vibration transmitting member having a through hole defined therein through which the optical fiber extends;
at least one piezoelectric element disposed on an outer circumferential surface of the vibration transmitting member and vibrating a distal end portion of the optical fiber via the vibration transmitting member;
a fixing member disposed on a proximal end side of the vibration transmitting member and holding the optical fiber in position; and
a tubular wire holding member having a plurality of wires integrally attached thereof and extending longitudinally therealong for supplying a voltage to the piezoelectric element, the wire holding member configured to cover at least a proximal end portion of the piezoelectric element wherein the tubular wire holding member has a contact portion securing the plurality of wires to an outer peripheral surface of the piezoelectric element in contact therewith,
the optical fiber scanner scanning light from the light source; and
a lens focusing light scanned by the optical fiber scanner.

7. An observation device comprising:
an illuminating device configured to apply illuminating light to a subject;
a light detector configured to detect returning light from the subject; and
a controller being used to electrically control the illuminating device wherein the illuminating device includes
a light source,
an optical fiber scanner configured for scanning light from the light source, and
a lens focusing the light scanned by the optical fiber scanner wherein
the optical fiber scanner having an optical fiber guiding light therethrough;
a vibration transmitting member configured to transmit vibrations to the optical fiber;
at least one piezoelectric element disposed on an outer circumferential surface of the vibration transmitting member and vibrating a distal end portion of the optical fiber via the vibration transmitting member,
a fixing member disposed on a proximal end side of the vibration transmitting member and holding the optical fiber in position, and
a tubular wire holding member having a plurality of wires integrally attached thereof and extending longitudinally therealong for supplying a voltage to the least one piezoelectric element, wherein the tubular wire holding member has a contact portion securing the plurality of wires to an outer peripheral surface of the piezoelectric element in contact therewith.

8. The observation device of claim 7, wherein the vibration transmitting member includes a through hole defined therein through which the optical fiber extends.

9. The observation device of claim 7, wherein the wire holding member configured to cover at least a proximal end portion of the piezoelectric element.

10. The observation device of claim 7, wherein the controller controls the light detector to detect the returning light in synchronism with a scanning period of the illuminating light and to generate an image of the subject by associating a detected intensity of the returning light with a scanning position of the illuminating light.
